(19) United States
(12) Patent Application Publication
(10) Pub. No.: US 2022/0290080 A1
(43) Pub. Date: Sep. 15, 2022

(54) CELL CULTURE ASSEMBLIES AND METHODS OF USING THE SAME

(71) Applicant: CORNING INCORPORATED, CORNING, NY (US)

(72) Inventor: William Joseph Lacey, North Andover, MA (US)

(21) Appl. No.: 17/635,867

(22) PCT Filed: Aug. 11, 2020
(86) PCT No.: PCT/US2020/045690

§ 371 Date: Feb. 16, 2022

Related U.S. Application Data
(60) Provisional application No. 62/887,975, filed on Aug. 16, 2019.

(51) Int. Cl. C12M 1/24; C12M 1/32

(52) U.S. Cl. CPC C12M 23/08; C12M 23/12

(57) ABSTRACT

A cell culture assembly includes a film defining a plurality of wells extending into the film, where each well of the plurality of wells defines a perimeter extending around the well, the plurality of wells defines an outer perimeter extending around the plurality of wells and extending between the perimeters of adjacent wells of the plurality of wells, and the outer perimeter extending around the plurality of wells defines a wave shape, and a sidewall member coupled to the film, where at least a portion of the sidewall member is oriented transverse to the film, the sidewall member including an engagement portion defining a wave shape that is complementary with the outer perimeter of the plurality of wells.

### CROSS REFERENCE TO RELATED APPLICATION

This application claims the benefit of priority under 35 U.S.C § 120 of U.S. Provisional Application Ser. No. 62/887,975 filed on Aug. 16, 2019, the content of which is relied upon and incorporated herein by reference in its entirety

### TECHNICAL FIELD

The disclosure generally relates to cell culture assemblies and methods of making and using the cell culture assemblies.

### BACKGROUND

Three dimensional (3D) cell culture relates to the growth of cells in an artificially-created environment that allows the cells to grow and/or interact primarily with each other in three dimensions. 3D cell culture assemblies provide an improvement over methods of growing cells in two dimensions (e.g., on a petri dish) as the 3D conditions more accurately model the in vivo environment.

For example, in two-dimensional cell culture systems, cells can attach to the substrate on which they are cultured. However, when cells are grown in three dimensions, such as spheroid wells, the cells primarily interact with each other rather than attaching to the substrate. Wells in conventional 3D cell culture systems are generally spaced apart from one another so that each well provides a discrete environment for growing cells. However, the spacing of the wells may limit the number of wells of the 3D cell culture system, thereby limiting the cells that can be grown in the cell culture system.

Accordingly, a need exists for alternative cell culture assemblies for maximizing the number of cells that can be grown in the cell culture assembly.

### SUMMARY

In one embodiment, a cell culture assembly includes a film defining a plurality of wells extending into the film, where each well of the plurality of wells defines a perimeter extending around the well, the plurality of wells defines an outer perimeter extending around the plurality of wells and extending between the perimeters of adjacent wells of the plurality of wells, and the outer perimeter extending around the plurality of wells defines a wave shape, and a sidewall member coupled to the film, where at least a portion of the sidewall member is oriented transverse to the film, the sidewall member including an engagement portion defining a wave shape that is complementary with the outer perimeter of the plurality of wells.

In another embodiment, a cell culture flask includes a plurality of sidewall members defining an interior space that is at least partially enclosed by the plurality of sidewall members, a film positioned within the interior space and defining a plurality of wells extending into the film, where each well of the plurality of wells defines a perimeter extending around the well, the plurality of wells defines an outer perimeter extending around the plurality of wells and extending between the perimeters of adjacent wells of the plurality of wells, and the outer perimeter extending around the plurality of wells defines a wave shape, and the film is coupled to at least one sidewall member of the plurality of sidewall members, where the at least one sidewall member is oriented transverse to the film, the at least one sidewall member including an engagement portion defining a wave shape that is complementary with the outer perimeter of the plurality of wells.

In another embodiment, a method for forming a cell culture assembly includes positioning a film on a coupling portion of a sidewall member, where the film defines a plurality of wells extending into the film, aligning crests of a plurality of crests extending outward from the sidewall member between adjacent wells of the plurality of wells such that each crest of the plurality of crests is positioned between adjacent wells of the plurality of wells, and coupling the film to the coupling portion of the sidewall member.

Additional features and advantages will be set forth in the detailed description which follows, and in part will be readily apparent to those skilled in the art from that description or recognized by practicing the embodiments as described herein, including the detailed description which follows, the claims, as well as the appended drawings.

It is to be understood that both the foregoing general description and the following detailed description are merely exemplary, and are intended to provide an overview or framework to understanding the nature and character of the claims. The accompanying drawings are included to provide a further understanding, and are incorporated in and constitute a part of this specification. The drawings illustrate embodiments, and together with the description serve to explain principles and operation of the various embodiments.

### BRIEF DESCRIPTION OF THE DRAWINGS

FIG. 1A schematically depicts a perspective view of a cell culture assembly, according to one or more embodiments shown and described herein;

FIG. 1B schematically depicts a front view of the cell culture assembly of FIG. 1A, according to one or more embodiments shown and described herein;

FIG. 1C schematically depicts a bottom view of the cell culture assembly of FIG. 1A, according to one or more embodiments shown and described herein;

FIG. 2 schematically depicts a top view of a film of the cell culture assembly of FIG. 1A, according to one or more embodiments shown and described herein;

FIG. 3 schematically depicts an enlarged top view of the film of FIG. 2, according to one or more embodiments shown and described herein;

FIG. 4 schematically depicts a top view of the film of FIG. 2 coupled to a sidewall member, according to one or more embodiments shown and described herein;

FIG. 5 schematically depicts a top view of the film of FIG. 2 coupled to another sidewall member, according to one or more embodiments shown and described herein;

FIG. 6 schematically depicts a section view of the film and sidewall member of FIG. 5, according to one or more embodiments shown and described herein;

FIG. 7 schematically depicts a section view of another film and sidewall member, according to one or more embodiments shown and described herein; and

FIG. 8 is a flowchart showing an example method for assembling the cell culture assembly of FIG. 1A.

### DETAILED DESCRIPTION

Cells cultured in three dimensions, such as spheroids, can exhibit more in vivo-like functionality than their counterparts cultured in two dimensions as monolayers. In two-dimensional cell culture systems, cells can attach to the substrate on which they are cultured. However, when cells are grown in three dimensions, such as within spheroids, the cells tend to interact with each other rather than attaching to the substrate. As such, cells cultured in three dimensions more closely resemble in vivo tissue in terms of cellular communication and the development of extracellular matrices. Spheroids thus provide a superior model for cell migration, differentiation, survival, and growth and therefore provide better systems for research, diagnostics, and drug efficacy, pharmacology, and toxicity testing.

In some embodiments, a film is provided that contains or comprises an array of microwells or wells. The film can form a part of a cell culture apparatus or device. For example, the film can form a part of a multiwell plate, a flask, a dish, a tube, a multi-layer cell culture flask, a bioreactor, or any other laboratory container intended to grow cells or spheroids. The microwells or wells (the term “microwell” and “well” are used interchangeably in this disclosure) are structured and arranged to provide an environment that is conducive to the formation of spheroids in culture. That is, in embodiments, the microwells have spheroid-inducing geometry. In addition, the wells are structured and arranged to provide for the movement of liquid into and out of the wells without trapping air between the substrate and liquid or liquid droplets that are introduced into the wells. That is, in embodiments, the microwells have capillary structures. For example, the wells in which cells are grown can be non-adherent to cells to cause the cells in the wells to associate with each other and form spheres. The spheroids expand to size limits imposed by the geometry of the wells. In some embodiments, the wells are coated with an ultra-low binding material to make the wells non-adherent to cells.

In some embodiments, the cell culture devices have frames comprising the footprint of the device, the substrate of which is configured such that cells cultured in the devices form spheroids. For example, the cell culture substrate in the devices is non-adherent to cells to cause the cells to associate with each other instead of the substrate. The cell culture substrate is further comprised of a plurality of microwells (or wells), the geometry of which enable cells grown in the wells to form similar-sized cell aggregates or spheroids. The spheroids expand to size limits imposed by the geometry of the microwells. In some embodiments, the wells have a low-binding treatment or are coated with an ultra-low binding material to make the wells non-adherent to cells.

Example non-adherent materials include perfluorinated polymers, olefins, or like polymers or mixtures thereof. Other examples include agarose, non-ionic hydrogels such as polyacrylamides, polyethers such as polyethylene oxide and polyols such as polyvinyl alcohol, or like materials or mixtures thereof. The combination of, for example, non-adherent wells, well geometry (e.g., size and shape), and/or gravity induce cells cultured in the wells to self-assemble into spheroids. Some spheroids maintain differentiated cell function indicative of a more in vivo-like response relative to cells grown in a monolayer. Other cells types, such as mesenchymal stromal cells, when cultured as spheroids retain their pluripotency,

In some embodiments, one or more cells are provided within the cell culture assembly. In some embodiments, the cells are cryopreserved. In some embodiments, the cells are in three-dimensional culture. In some such embodiments, the systems, devices, and methods comprise one or more spheroids. In some embodiments, one or more of the cells are actively dividing. In some embodiments, culture media are provided within the cell culture assembly for example and without limitation, nutrients (e.g., proteins, peptides, amino acids), energy (e.g., carbohydrates), essential metals and minerals (e.g., calcium, magnesium, iron, phosphates, sulphates), buffering agents (e.g., phosphates, acetates), indicators for pH change (e.g., phenol red, bromo-cresol purple), and/or selective agents (e.g., chemicals, antimicrobial agents, etc.).

A wide variety of cell types may be cultured. In some embodiments, a spheroid contains a single cell type. In some embodiments, a spheroid contains more than one cell type. In some embodiments, where more than one spheroid is grown, each spheroid is of the same type, while in other embodiments, two or more different types of spheroids are grown. Cells grown in spheroids may be natural cells or altered cells (e.g., cell comprising one or more non-natural genetic alterations). In some embodiments, the cell is a somatic cell. In some embodiments, the cell is a stem cell or progenitor cell (e.g., embryonic stem cell, induced pluripotent stem cell) in any desired state of differentiation (e.g., pluripotent, multi-potent, fate determined, immortalized, etc.). In some embodiments, the cell is a disease cell or disease model cell. For example, in some embodiments, the spheroid comprises one or more types of cancer cells or cells that can be induced into a hyper-proliferative state (e.g., transformed cells). Cells may be from or derived from any desired tissue or organ type, including but not limited to, adrenal, bladder, blood vessel, bone, bone marrow, brain, cartilage, cervical, corneal, endometrial, esophageal, gastrointestinal, immune system (e.g., T lymphocytes, B lymphocytes, leukocytes, macrophages, and dendritic cells), liver, lung, lymphatic, muscle (e.g., cardiac muscle), neural, ovarian, pancreatic (e.g., islet cells), pituitary, prostate, renal, salivary, skin, tendon, testicular, and thyroid. In some embodiments, the cells are mammalian cells (e.g., human, mice, rat, rabbit, dog, cat, cow, pig, chicken, goat, horse, etc.).

The cultured cells find use in a wide variety of research, diagnostic, drug screening and testing, therapeutic, and industrial applications.

In some embodiments, the cells are used for production of proteins or viruses. Three-dimensional culture allows for increased cell density, and higher protein yield per square centimeter of cell growth surface area. Any desired protein or viruses for vaccine production may be grown in the cells and isolated or purified for use as desired. In some embodiments, the protein is a native protein to the cells. In some embodiments, the protein is non-native. In some embodiments, the protein is expressed recombinantly. In some embodiments, the protein is overexpressed using a non-native promoter. The protein may be expressed as a fusion protein. In some embodiments, a purification or detection tag is expressed as a fusion partner to a protein of interest to facilitate its purification and/or detection. In some embodiments, fusions are expressed with a cleavable linker to allow separation of the fusion partners after purification.

In some embodiments, the protein is a therapeutic protein. Such proteins include, but are not limited to, proteins and peptides that replace a protein that is deficient or abnormal (e.g., insulin), augment an existing pathway (e.g., inhibitors or agonists), provide a novel function or activity, interfere with a molecule or organism, or deliver other compounds or proteins (e.g., radionuclides, cytotoxic drugs, effector proteins, etc.). In some embodiments, the protein is an immunoglobulin such as an antibody (e.g., monoclonal antibody) of any type (e.g., humanized, bi-specific, multi-specific, etc.). Therapeutic protein categories include, but are not limited to, antibody-based drugs, Fc fusion proteins, anticoagulants, antigens, blood factor, bone morphogenetic proteins, engineered protein scaffolds, enzymes, growth factors, hormones, interferons, interleukins, and thrombolytics. Therapeutic proteins may be used to prevent or treat cancers, immune disorders, metabolic disorders, inherited genetic disorders, infections, and other diseases and conditions.

In some embodiments, the protein is a diagnostic protein. Diagnostic proteins include, but are not limited to, antibodies, affinity binding partners (e.g., receptor-binding ligands), inhibitors, antagonists, and the like. In some embodiments, the diagnostic protein is expressed with or is a detectable moiety (e.g., fluorescent moiety, luminescent moiety (e.g., luciferase), colorimetric moiety, etc.).

In some embodiments, the protein is an industrial protein. Industrial proteins include, but are not limited to, food components, industrial enzymes, agricultural proteins, analytical enzymes, etc.

In some embodiments, the cells are used in drug discovery, characterization, efficacy testing, and toxicity testing. Such testing includes, but is not limited to, pharmacological effect assessment, carcinogenicity assessment, medical imaging agent characteristic assessment, half-life assessment, radiation safety assessment, genotoxicity testing, immunotoxicity testing, reproductive and developmental testing, drug interaction assessment, dose assessment, adsorption assessment, disposition assessment, metabolism assessment, elimination studies, etc. Specific cells types may be employed for specific tests (e.g., hepatocytes for liver toxicity, renal proximal tubule epithelial cells for nephrotoxicity, vascular endothelial cells for vascular toxicity, neuronal and glial cells for neurotoxicity, cardiomyocytes for cardiotoxicity, skeletal myocytes for rhabdomyolysis, etc.). Treated cells may be assessed for any number of desired parameters including, but not limited to, membrane integrity, cellular metabolite content, mitochondrial functions, lysosomal functions, apoptosis, genetic alterations, gene expression differences, and the like.

In some embodiments, the cell culture assemblies are a component of a larger system. In some embodiments, the system comprises a plurality (e.g., 2, 3, 4, 5, . . . , 10, . . . , 20, . . . , 50, . . . , 100, . . . , 1000, etc.) of such cell culture assemblies. In some embodiments, the system comprises an incubator for maintaining the culture devices at optimal culture conditions (e.g., temperature, atmosphere, humidity, etc.). In some embodiments, the system comprises detectors for imaging or otherwise analyzing cells. Such detectors include, but are not limited to, fluorimeters, luminometers, cameras, microscopes, plate readers (e.g., PERKIN ELMER ENVISION plate reader; PERKIN ELMER VIEWLUX plate reader), cell analyzers (e.g., GE IN Cell Analyzer 2000 and 2200; THERMO/CELLOMICS CELLNSIGHT High Content Screening Platform), and confocal imaging systems (e.g., PERKIN ELMER OPERAPHENIX high throughput content screening system; GE INCELL 6000 Cell Imaging System). In some embodiments, the system comprises perfusion systems or other components for supplying, re-supplying, and circulating culture media or other components to cultured cells. In some embodiments, the system comprises robotic components (e.g., pipettes, arms, plate movers, etc.) for automating the handing, use, and/or analysis of culture assemblies.

Embodiments described herein are directed to assemblies for culturing cells. More particularly, embodiments described herein are directed to cell culture assemblies including a film defining a plurality of wells and a sidewall member coupled to the film.

It is generally desirable to maximize the number of wells defined by the film, and accordingly, embodiments described herein include films having columns and rows of wells that are offset from one another to minimize a distance between adjacent wells. By minimizing the distance between adjacent wells, the number of wells on the film can be maximized. Moreover, by minimizing the distance between adjacent wells, the likelihood of cells growing within the wells instead of adhering to areas between adjacent wells may be maximized.

In some embodiments, the offset wells may form a wave-shaped perimeter. Cell culture assemblies described herein generally include sidewall members coupled to the film including engagement portions having a complementary and corresponding wave shape. By including engagement portions having complementary and corresponding shapes as the perimeter of the wells, a distance between the sidewall member and the wells may be minimized, thereby maximizing the likelihood of cells growing within the wells instead of adhering to areas of the film between the wells and the sidewall. These and other embodiments will now be described with relation to the appended drawings.

As referenced herein, the vertical direction refers to the upward-downward direction of the cell culture assembly and extends in the +/−Z-direction as depicted. The lateral direction, as referenced herein, refers to the cross-wise direction of the cell culture assembly and is transverse to the vertical direction, extending in the +/−X-direction as depicted. As referenced herein, the longitudinal direction refers to the lengthwise direction of the cell culture assembly and is transverse to the vertical and lateral directions, extending the +/−Y-direction as depicted.

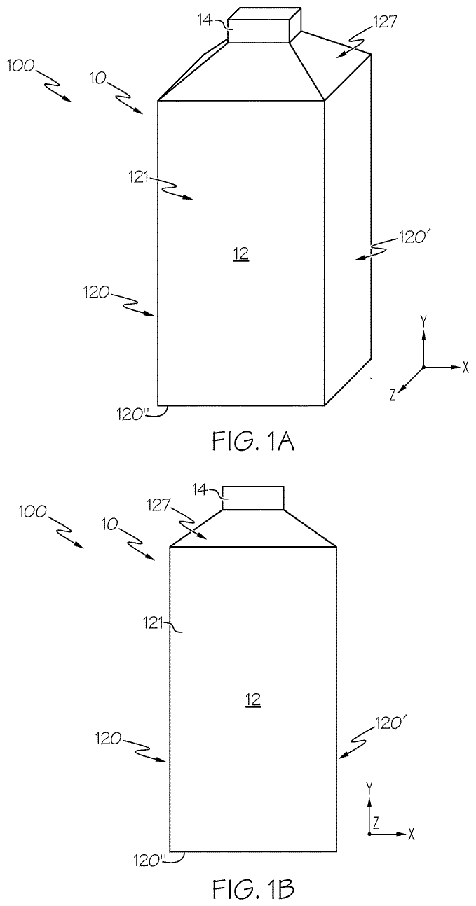

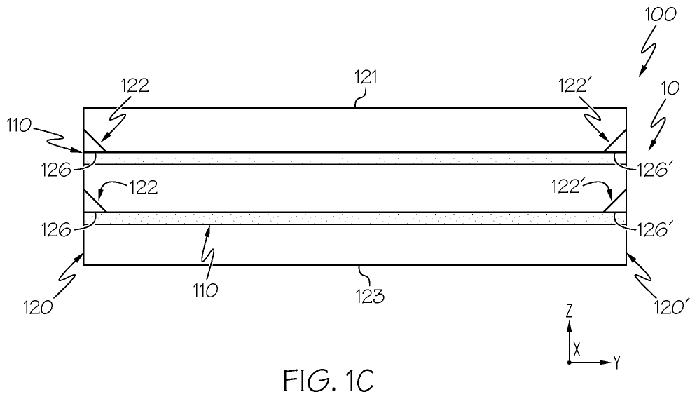

Referring to FIGS. 1A-1C, a perspective view, a front view, and a bottom view of a cell culture assembly 100 are schematically depicted, respectively. In the embodiment depicted in FIGS. 1A-1C, the cell culture assembly 100 is a cell culture flask 10 defining an interior space 12. The interior space 12 is generally bounded by a first sidewall member 120 and a second sidewall member 120′ positioned opposite the first sidewall member 120 in the lateral direction. The cell culture flask 10, in embodiments, further includes an end sidewall member 120″ extending between the first sidewall member 120 and the second sidewall member 120′, an upper wall 121, a bottom wall 123 positioned opposite the upper wall 121, and a top wall 127 positioned opposite the end sidewall member 120″. In embodiments, the top wall 127 defines an aperture allowing selective access to the interior space 12, and a cap 14 selectively positioned over the aperture. In the embodiment depicted in FIGS. 1A-1C, the cell culture flask 10 generally defines a rectangular prism, however, it should be understood that in embodiments, the cell culture flask 10 may define any suitable shape, for example and without limitation, a cylindrical shape or the like. Furthermore, while reference is made herein to cell culture flasks 10, it should be understood that cell culture assemblies 100 described herein may include any suitable assembly for growing cell cultures, for example and without limitation, microplates or the like.

In embodiments, the cell culture flask 10 generally includes one or more films 110 positioned at least partially within the interior space 12 of the cell culture flask 10. For example, in the embodiment depicted in FIG. 1C, the cell culture flask 10 includes a pair of films 110 that are spaced apart from one another in the vertical direction. While the embodiment depicted in FIG. 1C the cell culture flask 10 includes the pair of films 110, it should be understood that in embodiments, the cell culture flask 10 may include a single film 110 positioned at least partially within the interior space 12, or may include more than two films 110 positioned at least partially within the interior space 12.

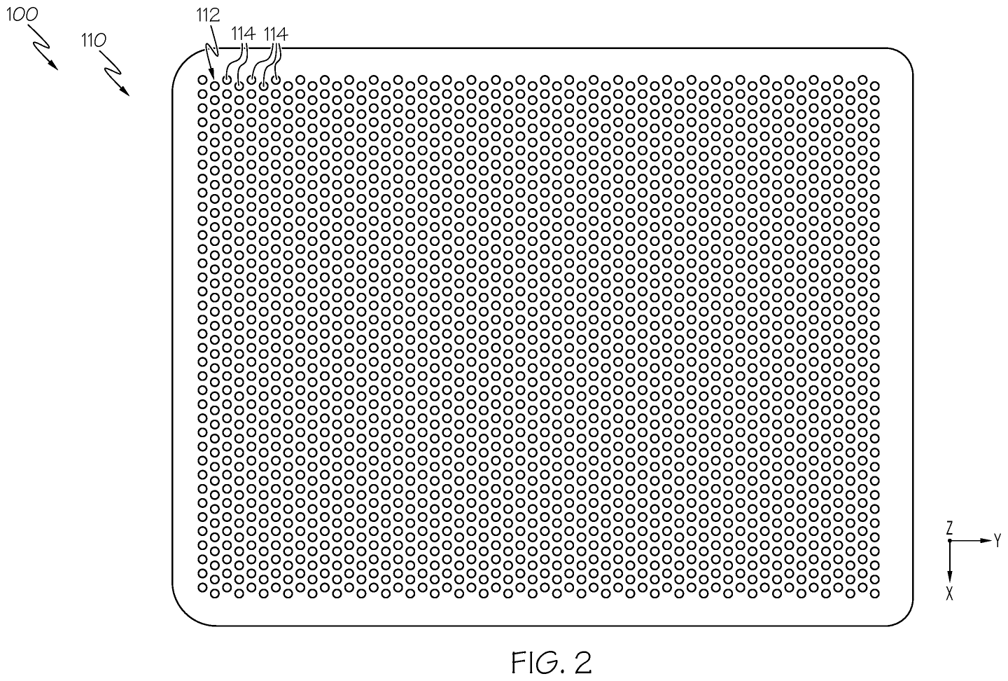

Referring to FIG. 2, a top view of one of the films 110 is schematically depicted. In embodiments, the film 110 defines a plurality of wells 112 extending into the film 110. In embodiments, individual wells 114 of the plurality of wells 112 each define a spheroidal shape. As noted above, by including a spheroidal shape, cells within the wells 114 can exhibit more in vivo-like functionality than their counterparts cultured in two dimensions as monolayers. In embodiments, the film 110 may be formed of any suitable material for facilitating a cell culture, such as polystyrene or the like.

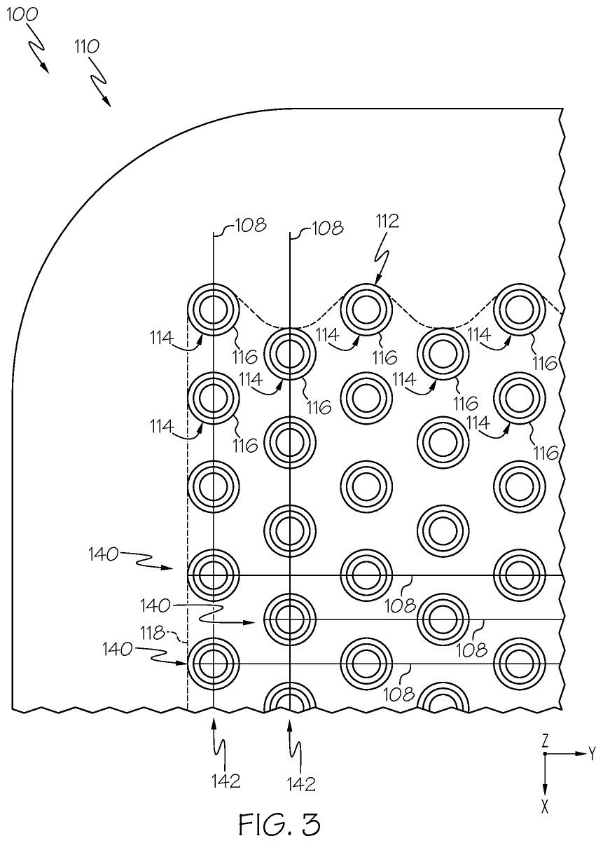

Referring to FIG. 3, an enlarged view of the wells 114 of the film 110 is schematically depicted. In embodiments, the wells 114 are arranged in a periodic pattern. For example, in the embodiment depicted in FIG. 3, the wells 114 are aligned in rows 140 extending in the longitudinal direction, and columns 142 extending in the lateral direction. To maximize the number of wells 114 positioned on the film 110 and minimize the space between the wells 114, adjacent rows 140 are offset from one another in the longitudinal direction, and adjacent columns 142 are offset from one another in the lateral direction. In particular, in embodiments, moving across the film 110 in the longitudinal direction (i.e., in the +Y-direction as depicted), a center 108 of adjacent wells 114 of the plurality of wells 112 are offset from one another in the lateral direction. Similarly moving across the film 110 in the lateral direction (i.e., in the +X-direction as depicted), the center 108 of adjacent wells 114 of the plurality of wells 112 are offset from one another in the longitudinal direction. By offsetting the wells 114, the density of the wells 114 on the film 110 may be increased, as compared to films in which the rows and columns of wells 114 are aligned with one another and areas of the film 110 positioned between the wells 114 may be minimized.

In embodiments, each well 114 of the plurality of wells 112 defines a perimeter 116 extending around the well 114. In the embodiment depicted in FIG. 3, each of the wells 114 include a spheroidal shape, and the perimeter 116 of each of the wells 114 includes a generally circular shape. The plurality of wells 112 defines an outer perimeter 118 extending around the plurality of wells 112 and extending between the perimeters 116 of adjacent wells 114. In embodiments in which the adjacent rows 140 and adjacent columns 142 of wells 114 are offset from one another, at least a portion of the outer perimeter 118 defines a wave shape. For example, in the embodiment depicted in FIG. 3, the outer perimeter 118 defines a wave shape extending in the longitudinal direction.

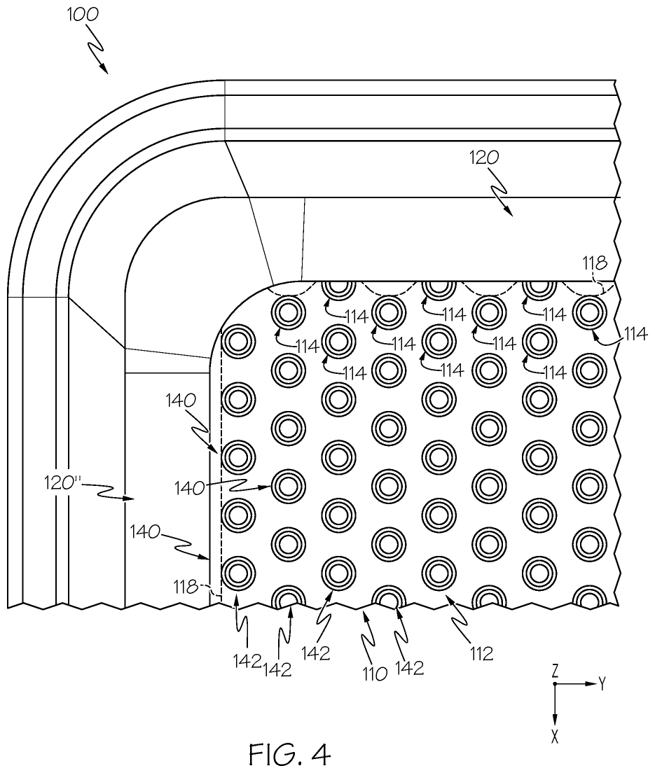

Referring to FIG. 4, in embodiments, the film 110 is coupled to one or more sidewall members. For example, in the view shown in FIG. 4, the first sidewall member 120 and the end sidewall member 120″ are depicted. While not shown in the enlarged view depicted in FIG. 4, the second sidewall member 120′ (FIG. 1C) may be substantially the same as the first sidewall member 120. In embodiments, the sidewall members 120, 120′ (FIG. 1C), 120″ may be formed of any suitable material for use in a cell culture assembly, such as polystyrene or the like, and may be formed of the same material or a different material than the film 110.

As described above, the sidewall members 120, 120′ (FIG. 1C), 120″ may form the walls of the cell culture flask 10 (FIG. 1C), and at least a portion of the sidewall members 120, 120′ (FIG. 1C), 120″ are oriented transverse to the film 110. At edges of the film 110 in which the outer perimeter 118 includes the wave shape, the sidewall member 120 may intersect and partially cover one or more of the wells 114, as shown in FIG. 4. By covering the one or more wells 114, the cells grown in the partially covered wells 114 may underperform or form an area of dead cells, reducing the efficacy of the film in growing viable cells.

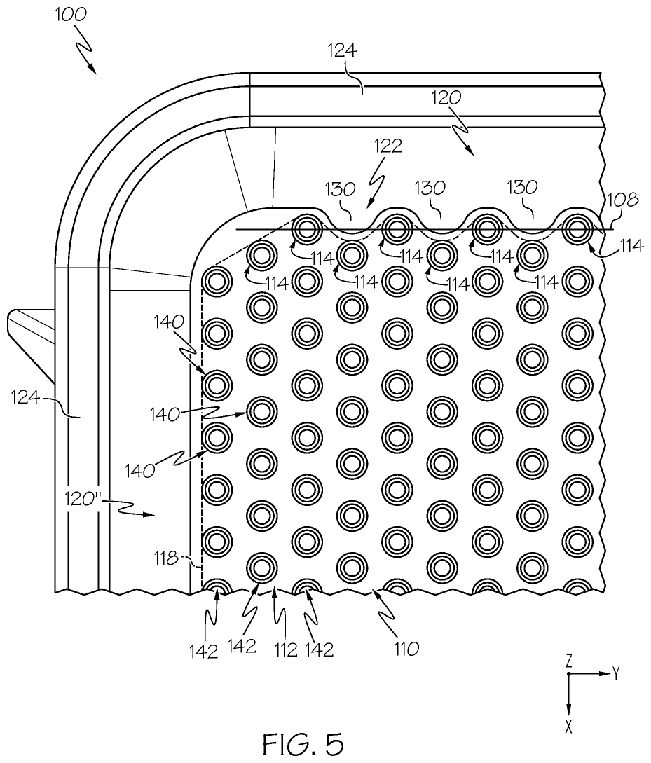

Referring to FIG. 5, at least one of the sidewall members 120, 120′ (FIG. 1C), 120″ includes an engagement portion 122 defining a wave shape that is complementary with the wave shape of the outer perimeter 118 of the plurality of wells 112. By forming a complementary wave shape as the outer perimeter 118 of the plurality of wells 112, the engagement portion 122 leaves the wells 114 uncovered, while minimizing distance between the sidewall member 120 and the plurality of wells 112, which may assist in reducing the number of cells that can become “stranded” between the sidewall member 120 and the plurality of wells 112.

In embodiments, the sidewall members 120 include a wall portion 124 that is oriented transverse to the film 110 (e.g., in the Z-direction as depicted). The engagement portion 122, in embodiments, includes a plurality of crests 130 extending outward from the wall portion 124. In some embodiments, each of the plurality of crests 130 extend outward beyond a center 108 of the wells 114 of the plurality of wells 112 that are adjacent to the crests 130. By extending beyond the center 108 of the wells 114 of the plurality of wells 112, the crests 130 of the engagement portion 122 may minimize a distance between the plurality of wells 112 and the sidewall member 120.

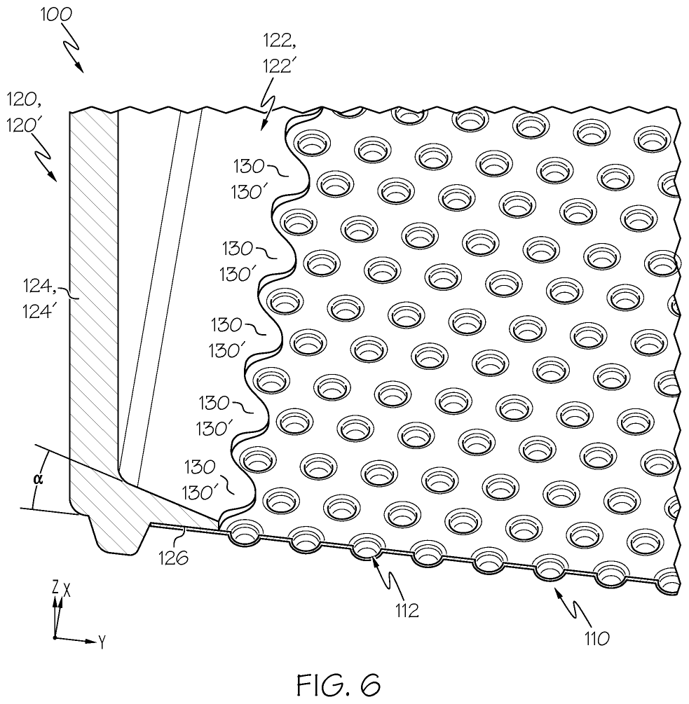

Referring to FIG. 6, a section view of the cell culture assembly 100 is depicted. In embodiments, the film 110 is coupled to the sidewall member 120. More particularly, in embodiments, the sidewall member 120 includes a coupling portion 126 that is oriented transverse to the wall portion 124, and the film 110 is coupled to the coupling portion 126 of the sidewall member 120. In embodiments, the coupling portion 126 defines a bottom surface of the engagement portion 122 and comprises the wave shape.

In some embodiments, the engagement portion 122 is oriented at an angle α with respect to the film 110. By orienting the engagement portion 122 at the angle α with respect to the film 110, cells deposited on the engagement portion 122 may be induced to flow onto the film 110, for example, under the influence of gravity. In embodiments, the engagement portion 122 of the sidewall member 120 extends in a plane that intersects the film 110 at least 5 degrees. In some embodiments, the engagement portion 122 of the sidewall member 120 extends in a plane that intersects the film 110 at between 15 and 30 degrees, inclusive of the endpoints. In some embodiments, the engagement portion 122 of the sidewall member 120 extends in a plane that intersects the film 110 at least 60 degrees.

While reference is made to the first sidewall member 120 with respect to FIG. 6, it should be understood that in embodiments, the second sidewall member 120′ is substantially the same as the first sidewall member 120. For example, the second sidewall 120′ comprising a second wall portion 124′ and a second engagement portion 122′ defining a wave shape that is complementary with the outer perimeter 118 (FIG. 5) of the plurality of wells 112. Furthermore, the second engagement portion 122′ includes a second plurality of crests 130′ extending outward from the second wall portion 124′.

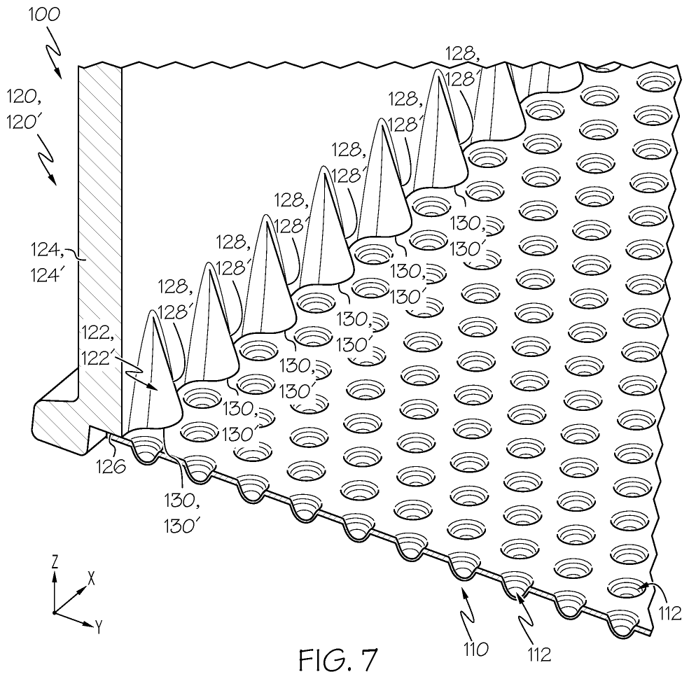

Referring to FIG. 7, in some embodiments, the engagement portion 122 of the sidewall member 120 includes a plurality of conical members 128. The conical members 128 generally extend upward along the wall portion 124 of the sidewall member 120 and define a conical shape. In embodiments, the conical members 128 define the plurality of crests 130 extending outward from the wall portion 124. The conical shape of the conical members 128 also includes a taper that further assists in inducing flow of cells onto the film 110, and more particularly into the plurality of wells 112. In these embodiments, the conical members 128 may extend in a plane that intersects the film 110 at a comparatively high angle. For example in some embodiments, the conical members may intersect the film 110 at about 75 degrees. In some embodiments, the conical members 128 extend in a plane that intersects the film 110 at least 5 degrees. In some embodiments, the conical members 128 extend in a plane that intersects the film 110 at between 15 and 30 degrees, inclusive of the endpoints. In some embodiments, the conical members 128 extend in a plane that intersects the film 110 at least 60 degrees.

While reference is made to the first sidewall member 120 with respect to FIG. 7, it should be understood that in embodiments, the second sidewall member 120′ is substantially the same as the first sidewall member 120. For example, the second sidewall 120′ comprising a second wall portion 124′ and a second engagement portion 122′ defining a wave shape that is complementary with the outer perimeter 118 (FIG. 5) of the plurality of wells 112. Furthermore, the second engagement portion 122′ includes a second plurality of conical members 128 defining a second plurality of crests 130′ extending outward from the second wall portion 124′.

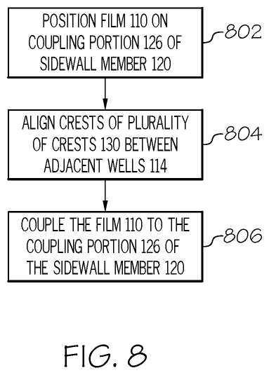

Referring to FIGS. 1C, 7, and 8, a method for forming a cell culture assembly 100 is schematically depicted. In a first step 802, the film 110 is positioned on the coupling portion 126 of the sidewall member 120. With the film 110 positioned on the coupling portion 126 of the sidewall member 120, at step 804, the method includes aligning crests of the plurality of crests 130 between adjacent wells 114 of the plurality of wells 112, such that each of the crests of the plurality of crests 130 are positioned between adjacent wells 114 of the plurality of wells 112. At step 806, the method includes coupling the film 110 to the coupling portion 126 of the sidewall member 120. In embodiments, the film 110 is coupled to the coupling portion 126 of the sidewall through any suitable methodology, such as adhesives, mechanical fasteners, plastic welding or the like.

In embodiments, the sidewall member 120 is a first sidewall member, and the cell culture assembly 100 further includes the second sidewall member 120′ positioned opposite the first sidewall member 120. In these embodiments, the method further includes positioning the film 110 on a second coupling portion 126′ of the second sidewall member 120′. The method, in embodiments, may further include aligning crests of a second plurality of crests 130′ extending outward from the second sidewall member 120′ between adjacent wells 114 of the plurality of wells 112 such that each crest of the second plurality of crests 130′ are positioned between adjacent wells 114 of the plurality of wells 112. In embodiments, the method further includes coupling the film 110 to the second coupling portion 126′ of the second sidewall member 120′.

Accordingly, it should now be understood that embodiments described herein are generally directed assemblies for culturing cells. More particularly, embodiments described herein are directed to cell culture assemblies including a film defining a plurality of wells and a sidewall member coupled to the film. Embodiments described herein include films having columns and rows of wells that are offset from one another to minimize a distance between adjacent wells. By minimizing the distance between adjacent wells, the number of wells on the film can be maximized. Moreover, by minimizing the distance between adjacent wells, the likelihood of cells growing within the wells instead of adhering to areas between adjacent wells may be maximized.

In some embodiments, the offset wells form a wave-shaped perimeter. Cell culture assemblies described herein generally include sidewall members coupled to the film including engagement portions having a complementary and corresponding wave shape. By including engagement portions having complementary and corresponding shapes as the perimeter of the wells, a distance between the sidewall member and the wells may be minimized, thereby maximizing the likelihood of cells growing within the wells instead of adhering to areas of the film between the wells and the sidewall.

Embodiments can be described with reference to the following numbered clauses, with preferred features laid out in the dependent clauses:

1. A cell culture assembly comprising a film defining a plurality of wells extending into the film, wherein each well of the plurality of wells defines a perimeter extending around the well; the plurality of wells defines an outer perimeter extending around the plurality of wells and extending between the perimeters of adjacent wells of the plurality of wells; and the outer perimeter extending around the plurality of wells defines a wave shape; and a sidewall member coupled to the film, wherein at least a portion of the sidewall member is oriented transverse to the film, the sidewall member comprising an engagement portion defining a wave shape that is complementary with the outer perimeter of the plurality of wells.

2. The cell culture assembly of the preceding clause, wherein each of the plurality of wells define a spheroidal shape.

3. The cell culture assembly of either clauses 1 or 2, wherein the engagement portion of the sidewall member extends in a plane that intersects the film at least 5 degrees.

4. The cell culture assembly of any of the preceding clauses, wherein the engagement portion of the sidewall member comprises a plurality of conical members.

5. The cell culture assembly of any of the preceding clauses, wherein the sidewall member defines a wall portion oriented transverse to the film, and a coupling portion oriented transverse to the wall portion, wherein the film is coupled to the coupling portion of the sidewall member.

6. The cell culture assembly of clause 5, wherein the coupling portion defines a bottom surface of the engagement portion and comprises the wave shape.

7. The cell culture assembly of any of the preceding clauses, wherein the sidewall member is a first sidewall member, and the cell culture assembly further comprise a second sidewall member positioned opposite the first sidewall member, the second sidewall member comprising a second engagement portion defining a wave shape that is complementary with the outer perimeter of the plurality of wells.

8. The cell culture assembly of any of the preceding clauses, wherein the sidewall member defines a wall portion oriented transverse to the film, and the engagement portion comprises a plurality of crests extending outward from the wall portion, wherein the each of the plurality of crests extends outward beyond a center of wells of the plurality of wells adjacent to the plurality of crests.

9. A cell culture flask comprising a plurality of sidewall members defining an interior space that is at least partially enclosed by the plurality of sidewall members; a film positioned within the interior space and defining a plurality of wells extending into the film, wherein each well of the plurality of wells defines a perimeter extending around the well; the plurality of wells defines an outer perimeter extending around the plurality of wells and extending between the perimeters of adjacent wells of the plurality of wells; and the outer perimeter extending around the plurality of wells defines a wave shape; and the film is coupled to at least one sidewall member of the plurality of sidewall members, wherein the at least one sidewall member is oriented transverse to the film, the at least one sidewall member comprising an engagement portion defining a wave shape that is complementary with the outer perimeter of the plurality of wells.

10. The cell culture flask of clause 9, wherein each of the plurality of wells define a spheroidal shape.

11. The cell culture flask of either clauses 9 or 10, wherein the engagement portion of the at least one sidewall member extends in a plane that intersects the film at least 5 degrees.

12. The cell culture flask of any of clauses 9-11, wherein the engagement portion of the at least one sidewall member comprises a plurality of conical members.

13. The cell culture flask of any of clauses 9-12, wherein the at least one sidewall member defines a wall portion oriented transverse to the film, and a coupling portion oriented transverse to the wall portion, wherein the film is coupled to the coupling portion of the at least one sidewall member.

14. The cell culture flask of clause 13, wherein the coupling portion extends to a bottom surface of the engagement portion and comprises the wave shape.

15. The cell culture flask of any of clauses 9-14, wherein the at least one sidewall member defines a wall portion oriented transverse to the film, and the engagement portion comprises a plurality of crests extending outward from the wall portion, wherein the each of the plurality of crests extends outward beyond a center of wells of the plurality of wells adjacent to the plurality of crests.

16. The cell culture flask of any of clauses 9-15, wherein the film is a first film, and the cell culture flask further comprises a second film spaced apart from the first film.

17. A method for forming a cell culture assembly, the method comprising positioning a film on a coupling portion of a sidewall member, wherein the film defines a plurality of wells extending into the film; aligning crests of a plurality of crests extending outward from the sidewall member between adjacent wells of the plurality of wells such that each crest of the plurality of crests is positioned between adjacent wells of the plurality of wells; and coupling the film to the coupling portion of the sidewall member.

18. The method of clause 17, wherein the sidewall member is a first sidewall member and the method further comprises positioning the film on a second coupling portion of a second sidewall member positioned opposite the first sidewall member.

19. The method of clause 18, further comprising aligning crests of a second plurality of crests extending outward from the second sidewall member between adjacent wells of the plurality of wells such that each crest of the second plurality of crests are positioned between adjacent wells of the plurality of wells.

20. The method of clause 19, further comprising coupling the film to the second coupling portion of the second sidewall member.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred, in any respect. This holds for any possible non-express basis for interpretation, including: matters of logic with respect to arrangement of steps, operational flow, order of components, or orientation of components; plain meaning derived from grammatical organization or punctuation, and; the number or type of embodiments described in the specification.

It will be apparent to those skilled in the art that various modifications and variations can be made without departing from the spirit or scope of the disclosure. Since modifications, combinations, sub-combinations and variations of the disclosed embodiments incorporating the spirit and substance of the disclosure may occur to persons skilled in the art, the disclosure should be construed to include everything within the scope of the appended claims and their equivalents.

What is claimed is:

1. A cell culture assembly comprising:
a film defining a plurality of wells extending into the film, wherein:
each well of the plurality of wells defines a perimeter extending around the well;
the plurality of wells defines an outer perimeter extending around the plurality of wells and extending between the perimeters of adjacent wells of the plurality of wells; and
the outer perimeter extending around the plurality of wells defines a wave shape; and
a sidewall member coupled to the film, wherein at least a portion of the sidewall member is oriented transverse to the film, the sidewall member comprising an engagement portion defining a wave shape that is complementary with the outer perimeter of the plurality of wells.

2. The cell culture assembly of claim 1, wherein each of the plurality of wells define a spheroidal shape.

3. The cell culture assembly of claim 1, wherein the engagement portion of the sidewall member extends in a plane that intersects the film at least 5 degrees.

4. The cell culture assembly of claim 1, wherein the engagement portion of the sidewall member comprises a plurality of conical members.

5. The cell culture assembly of claim 1, wherein the sidewall member defines a wall portion oriented transverse to the film, and a coupling portion oriented transverse to the wall portion, wherein the film is coupled to the coupling portion of the sidewall member.

6. The cell culture assembly of claim 5, wherein the coupling portion defines a bottom surface of the engagement portion and comprises the wave shape.

7. The cell culture assembly of claim 1, wherein the sidewall member is a first sidewall member, and the cell culture assembly further comprise a second sidewall member positioned opposite the first sidewall member, the second sidewall member comprising a second engagement portion defining a wave shape that is complementary with the outer perimeter of the plurality of wells.

8. The cell culture assembly of claim 1, wherein the sidewall member defines a wall portion oriented transverse to the film, and the engagement portion comprises a plurality of crests extending outward from the wall portion, wherein the each of the plurality of crests extends outward beyond a center of wells of the plurality of wells adjacent to the plurality of crests.

9. A cell culture flask comprising:
a plurality of sidewall members defining an interior space that is at least partially enclosed by the plurality of sidewall members;
a film positioned within the interior space and defining a plurality of wells extending into the film, wherein:
each well of the plurality of wells defines a perimeter extending around the well;
the plurality of wells defines an outer perimeter extending around the plurality of wells and extending between the perimeters of adjacent wells of the plurality of wells; and
the outer perimeter extending around the plurality of wells defines a wave shape; and
the film is coupled to at least one sidewall member of the plurality of sidewall members, wherein the at least one sidewall member is oriented transverse to the film, the at least one sidewall member comprising an engagement portion defining a wave shape that is complementary with the outer perimeter of the plurality of wells.

10. The cell culture flask of claim 9, wherein each of the plurality of wells define a spheroidal shape.

11. The cell culture flask of claim 9, wherein the engagement portion of the at least one sidewall member extends in a plane that intersects the film at least 5 degrees.

12. The cell culture flask of claim 9, wherein the engagement portion of the at least one sidewall member comprises a plurality of conical members.

13. The cell culture flask of claim 9, wherein the at least one sidewall member defines a wall portion oriented transverse to the film, and a coupling portion oriented transverse to the wall portion, wherein the film is coupled to the coupling portion of the at least one sidewall member.

14. The cell culture flask of claim 13, wherein the coupling portion extends to a bottom surface of the engagement portion and comprises the wave shape.

15. The cell culture flask of claim 9, wherein the at least one sidewall member defines a wall portion oriented transverse to the film, and the engagement portion comprises a plurality of crests extending outward from the wall portion, wherein the each of the plurality of crests extends outward beyond a center of wells of the plurality of wells adjacent to the plurality of crests.

16. The cell culture flask of claim 9, wherein the film is a first film, and the cell culture flask further comprises a second film spaced apart from the first film.

17. A method for forming a cell culture assembly, the method comprising:
positioning a film on a coupling portion of a sidewall member, wherein the film defines a plurality of wells extending into the film;
aligning crests of a plurality of crests extending outward from the sidewall member between adjacent wells of the plurality of wells such that each crest of the plurality of crests is positioned between adjacent wells of the plurality of wells; and
coupling the film to the coupling portion of the sidewall member.

18. The method of claim 17, wherein the sidewall member is a first sidewall member and the method further comprises positioning the film on a second coupling portion of a second sidewall member positioned opposite the first sidewall member.

19. The method of claim 18, further comprising aligning crests of a second plurality of crests extending outward from the second sidewall member between adjacent wells of the plurality of wells such that each crest of the second plurality of crests are positioned between adjacent wells of the plurality of wells.

20. The method of claim 19, further comprising coupling the film to the second coupling portion of the second sidewall member.
